(19) United States
(12) Patent Application Publication
(10) Pub. No.: US 2013/0203059 A1
(43) Pub. Date: Aug. 8, 2013

(54) Method for Diagnosis of Bladder Cancer and Related Kits

(71) Applicants: Antonio Feliciello, Parete (IT); Luigi Insabato, Napoli (IT)

(72) Inventors: Antonio Feliciello, Parete (IT); Luigi Insabato, Napoli (IT)

(73) Assignee: Topogen, Inc., Port Orange, FL (US)

(21) Appl. No.: 13/808,000

(22) PCT Filed: Jun. 28, 2011
(86) PCT No.: PCT/EP2011/060788

§ 371 Date: Apr. 15, 2013

Related U.S. Application Data
(60) Provisional application No. 61/361,172, filed on Jul. 2, 2010.

(51) Int. Cl. C12Q 1/42; C12Q 1/68; G01N 33/68

(52) U.S. Cl. CPC C12Q 1/42; G01N 33/6893; C12Q 1/6886
USPC 435/6.11; 435/7.4; 435/21; 435/6.12

(57) ABSTRACT

The invention refers to a novel molecular biomarker, namely PTPD1, that is markedly increased in human bladder cancers. PTPD1 expression positively correlated with the grading and invasiveness potential of these tumors. PTPD1 can be detected at high levels in exfoliated bladder cells isolated from urine of bladder cancer patients, while no PTPD1 signal was evident in normal exfoliated bladder cells. Thus, PTPD1 detection in urine samples may represent a novel and reliable marker for non-invasive diagnosis of aggressive bladder cancer.

### FIELD OF THE INVENTION

The authors have discovered a novel molecular biomarker, namely PTPD1, that is markedly increased in human bladder cancers. PTPD1 expression positively correlated with the grading and invasiveness potential of these tumors. Moreover, the authors found that PTPD1 can be detected at high levels in exfoliated bladder cells isolated from urine of bladder cancer patients, while no PTPD1 signal was evident in normal exfoliated bladder cells. Thus, PTPD1 detection in urine samples may represent a novel and reliable marker for non-invasive diagnosis of aggressive bladder cancer.

### BACKGROUND ART

Bladder cancer is among the most common cancers in Western countries and contributes significantly to overall cancer mortality. The probability of recurrence is in excess of 50%, and stage/grade progression occurs in 10% to 50% of cases. Treatment of invasive urothelial carcinomas is ineffective. 50% of patients die from metastases within 2 years of diagnosis, and the 5-year survival rate for metastatic bladder cancer is 6% (31). Low-grade papillary tumors, which rarely become muscle-invasive, and high-grade invasive tumors, which generally become metastatic, appear to arise by different mechanisms.

Diagnosis and staging of urothelial carcinomas presents problems. Cystoscopy is accurate (ca. 91%), but carcinoma in situ and other cancers can escape detection. It is also quite expensive, since lifelong follow-ups are required. Cytology using bladder wash or voided urine samples is unreliable. Some success in diagnosis has been reported testing for the nuclear matrix protein, NMP22, in urine. However, in general, prognostic indicators for bladder cancer are problematic. All of the urine-based biomarkers—other than cytology- can result in false-positives and false-negatives; there is no biomarker that can be used a universal test for bladder cancer. Although certain markers, such as Ki-67 and p53, appear to be promising in predicting recurrence and progression of bladder cancer, the data are still heterogeneous. Clearly, there is a need for improved detection systems.

Tyrosine-protein phosphatase non-receptor type 21 (PTPN21; NP_{—}008970; gene synonym PTPD1, PTPRL10, SEQ ID No. 1) is a cytosolic non-receptor tyrosine phosphatase expressed in several tissues (Moller et al., 1994; Corner et al., 1998; Cardone et al., 2004). PTPD1 cDNA (PTPN21; NM_{—}007039) encodes a protein of 1174 amino acids (SEQ ID No. 1) containing an N-terminal sequence homologous to the Four-point-one Ezrin-Radixin-Moesin (FERM domain) protein family, which includes PTPH1 and PTPMEG1. The FERM motif is a modular structure present within a family of peripheral membrane proteins that link the cytoskeleton to the plasma membrane. The catalytic domain (PTP) is positioned at the extreme C-terminus of PTPD1. An intervening sequence of about 580 residues without homology to known proteins separates the ezrin-like and the PTP domains. Furthermore, PTPD1 contains SH2 and SH3 binding domains that may serve as a molecular platform for several signal transduction molecules (FIG. 1).

### DESCRIPTION OF THE INVENTION

Authors of the instant invention found that:
1. PTPD1 is overexpressed in human bladder cancer tissues. PTPD1 has been implicated in critical aspects of cell-cell communication, growth and motility. This led us to investigate the expression profile of PTPD1 in human bladder epithelial cancer with high recurrence rate and metastatic behavior. The risk grade of cancer lesions was assessed basing upon the categorization provided by the WHO Bladder grading. Tissues samples were derived from first biopsies of patients affected by urothelium hyperplasia, urothelial papilloma, low malignant potential urothelial neoplasia, low grade or high grade urothelial carcinoma. By immunoblot analysis and immunohistochemistry, authors found that PTPD1 is nearly undetectable in normal tissue, in hyperplastic urothelium and urothelial papilloma, while low-moderate levels are present in low-grade urothelial carcinoma. In contrast, PTPD1 strongly accumulated in samples derived from high-grade urothelial carcinomas. The analysis was carried out on a total of 46 patients: 12 normal or hyperplastic urothelium, 3 urothelial papilloma, 15 low grade urothelial carcinoma and 19 cases of high grade urothelial carcinoma. Data on PTPD1 overexpression in bladder cancer specimens was confirmed in a large number (about 500) of human bladder cancers by tissue microarray analysis (TMA).
2. PTPD1 is detected of in exfoliated bladder cancer cells. Authors analyzed PTPD1 levels in exfoliated bladder cells recovered from urine samples. Cells from urine specimens of control and bladder cancer patients were subjected to immunocytochemistry using anti-PTPD1 antibody generated by authors using standard techniques. The data demonstrate that PTPD1 was nearly undetectable in exfoliated, normal bladder cells from healthy volunteers. In contrast, urine cancer cells from bladder patients show a strong PTPD1 immunoreactive signal, compared to normal cells present in the same urine specimen where PTPD1 staining was undetectable. Thus, PTPD1 detection in urine samples represents a novel and reliable marker for non-invasive diagnosis of aggressive bladder cancer.
PTPD1 detection may be achieved with techniques known to the skilled person, as immunodetection of the protein itself or of fragments thereof, enzymatic assays, PTPD1 RNA detection.

It is therefore an object of the invention a method for the diagnosis or the monitoring control for therapy of bladder cancer characterized in detecting the PTPD1 protein or an immunological fragment thereof, or the PTPD1 enzymatic activity, or the PTPD1 mRNA in a body sample.

The PTPD1 protein is a protein having essentially the sequence of SEQ ID No. 1 or an allelic variant thereof.

Preferably the detecting of the PTPD1 protein or of an immunological fragment thereof is performed by:—allowing the body sample to react with a PTPD1 protein specific ligand to form a complex;—detecting the complex. More preferably said specific ligand is an anti-PTPD1 antibody (monoclonal or polyclonal) or an immunological, synthetic or recombinant derivative thereof.

The anti-PTPD1 antibody or immunological, synthetic or recombinant derivative thereof is obtainable by using as immunogen the whole PTPD1 protein of SEQ ID No. 1 or an immunogenic fragment thereof. Preferably the immunogenic fragment of the PTPD1 protein of SEQ ID No. 1 is comprised between aa 751 and aa 910 of SEQ ID No. 1. Alternatively the immunogenic fragment of the PTPD1 protein of SEQ ID No. 1 consists of a sequence between aa 751 and aa 910 of SEQ ID No. 1.

In a preferred embodiment the detecting step is by means of detection of a specific fluorescent signal.

In an alternative preferred embodiment of the method of the invention the detecting of the PTPD1 enzymatic activity is performed by fluorescent or radiolabeled assays, i.e. based on PTPD1-catalyzed release of the phosphate group from a given substrate.

In an alternative preferred embodiment of the method of the invention the detecting of the PTPD1 mRNA is performed by Northern blot analysis, polymerase chain reaction (PCR) or PCR-derived methods, or nucleic acid amplification based methods.

The method of the invention is performed on a body sample out of the body, as a bladder tissue, or body fluid or a fraction thereof, i.e. urine or its sediment. It is a further object of the invention a bladder cancer diagnostic kit comprising:
a solid phase adhered PTPD1 protein specific ligand as above disclosed;
a detection ligand able to bind to PTPD1 protein specific ligand—PTPD1 protein. Preferably the PTPD1 protein specific ligand is an anti-PTPD1 antibody or a derivative thereof, as above disclosed, more preferably said anti-PTPD1 antibody or derivative thereof is obtained by using a fragment of the PTPD1 protein as antigen, more preferably in the region between aa. 618 and aa. 910 of the PTPD1 protein.

### EXAMPLES

### Figure Legends

FIG. 1. A. Schematic representation of the human PTPD1 protein. PTP, catalytic domain; AcR, acidic region; FERM, Four point one-Ezrin-Radixin-Moesin domain Binding domains for src, actin and FAK are indicated.

FIG. 2. PTPD1 is highly expressed in bladder carcinomas. A-B. Tumor samples (T) were isolated from patients affected by high grade (lanes #2, #3, #4, #6, #7, #8) or low grade (lanes #1, #5, #9) urothelial carcinoma. Normal tissue (N) surrounding each neoplastic lesion was also isolated. Tissue samples were lysed, resolved on 8% SDS-PAGE gels and immunoblotted with the following antibody: anti-peptide PTPD1 (ab1) (A) or anti-polypeptide PTPD1 (ab2) (B), anti-ERK2 and anti-cytokeratins. C. Tissue sections from normal bladder (a), hyperplastic bladder (b and c) and high grade (d) of urothelial carcinoma were immunostained with anti-PTPD1 antibody and analyzed by light microscopy. Higher resolution panels (a′, b′, c′, d′) of each set of images are shown on the right. D. Bladder lesions were subgrouped in three categories: a. normal/hyperplastic; b. low grade urothelial carcinoma; c. high grade urothelial carcinoma. Cumulative data and relative abundance of PTPD1 in each category are shown.

FIG. 3. Tissue Microarray Analysis (TMA) for PTPD1 expression in human bladder biopsies. Tissue microarray of 505 bladder samples ranging from normal tissue, benign lesions and urothelial carcinomas was immunostained with anti-PTPD1 polyclonal antibody. A. Enlarged section of representative biopsies of normal and cancer lesions immunostained with anti-PTPD1 antibody. B. Cumulative data are expressed as percentage of PTPD1-positive samples within the two main categories (normal/hyperplastic lesions and urothelial carcinomas). P value is indicated on the right column C. PTPD1-positive urothelial carcinomas were scored for Ki-67 positivity. Cut-off value represents the percentage of Ki-67 positive cells versus total cells scored. D. Inverse correlation between bladder stage disease (pTa, pT1 and pT3) and PTPD1 signal. The analysis was carried out on a total of 349 patients with urothelial carcinoma.

FIG. 4. PTPD1 is present in urinary exfoliated bladder cells. Urine samples from control (A) and bladder cancer patient (B) were subjected to immunocytochemistry using anti-PTPD1 antibody. A representative experiment of PTPD1 detection in urine samples from bladder cancer patients (#5) is shown. Enlarged section of the panel B is also shown (B′). Arrows indicate normal and urothelial cancer cells.

### MATERIALS AND METHODS

Human bladder samples. Tissue samples were isolated from patients affected by benign and malignant tumors of the urinary bladder, along with patients affected by hyperplastic or normal urothelial mucosa were retrieved from the files of the Department of Biomorphological and Functional Sciences, Pathology Section, and Department of Urology, University “Federico II” of Naples, Italy. The risk grade was assessed basing upon the categorization provided by the WHO Bladder grading 2004.

Anti-PTPD1 polyclonal antibodies, Anti-PTPD1 polyclonal antibodies were generated as follows as already disclosed (Carlucci et al. 2008). A cDNA encoding for the central core of human PTPD1 protein (aa. 751-910) was subcloned in pRSET-vector, expressed in BL21 bacteria and affinity purified on column The purified fragment was used to immunize rabbits. The specificity of the antibody was tested by western blot and immunofluorescence (Carlucci et al., 2008). Immune anti-PTPD1 IgG were also produced.

Immunohistochemistry. Formalin-fixed, paraffin embedded tissues from patients subjected to cystoscopic biopsy were selected. Representative slides of each tumor were stained with hematoxylin and eosin to confirm the diagnosis and grading of the tumors. 4-μm serial sections from representative blocks were cut, mounted on poly-L-lysine coated glass slides and used for the immunohistochemical examination. Two polyclonal antibodies raised against distinct domains (residues 751-910; residues 618-631) of human PTPD1 protein (SEQ ID No. 1) were used. The antibody raised against residues 618-631 was previously described (Moller et al., 1994). Both anti-PTPD1 antibodies gave identical immunostaining pattern. Sections were de-paraffined in xylene and rehydrated through a decreasing concentration of alcohol to water. Before incubation with the antibodies the slides were heated in a pression cooker for 3 minutes in a solution of 0.01 mol/L sodium citrate (pH 6.0). To avoid non-specific binding, sections were pre-incubated with non-immune serum (1:20, Dakopatts, Hamburg, Germany) diluted in PBS/BSA, 1%, for 25 minutes, at room temperature. Endogenous peroxidases activity was reduced by incubation with 3% hydrogen peroxide for 20 minutes. Representative sections were incubated with the listed primary antibodies, overnight at 4° C. Subsequently, the slides were incubated with biotinylated secondary antibodies, peroxidase-labelled streptavidin (DAKO LSAB kit HRP, Carpinteria, Calif.) and chromogenic substrate diaminobenzidine (DAB, Vector Laboratories, Burlingame, U.S.A.) for the development of the peroxidase activity. Slides were counterstained with hematoxylin, dehydrated and cover-slipped with a synthetic mounting medium (Entellan, Merck, Germany). Omission of primary antibody and substitution with phosphate-buffered saline were used as negative controls. Section analysis was performed by two pathologists blind to the histological typing and to the follow-up data of the single cases of bladder carcinoma. Only cells with a definite membrane and cytoplasmic staining were judged as positive for each antibody.

### RESULTS

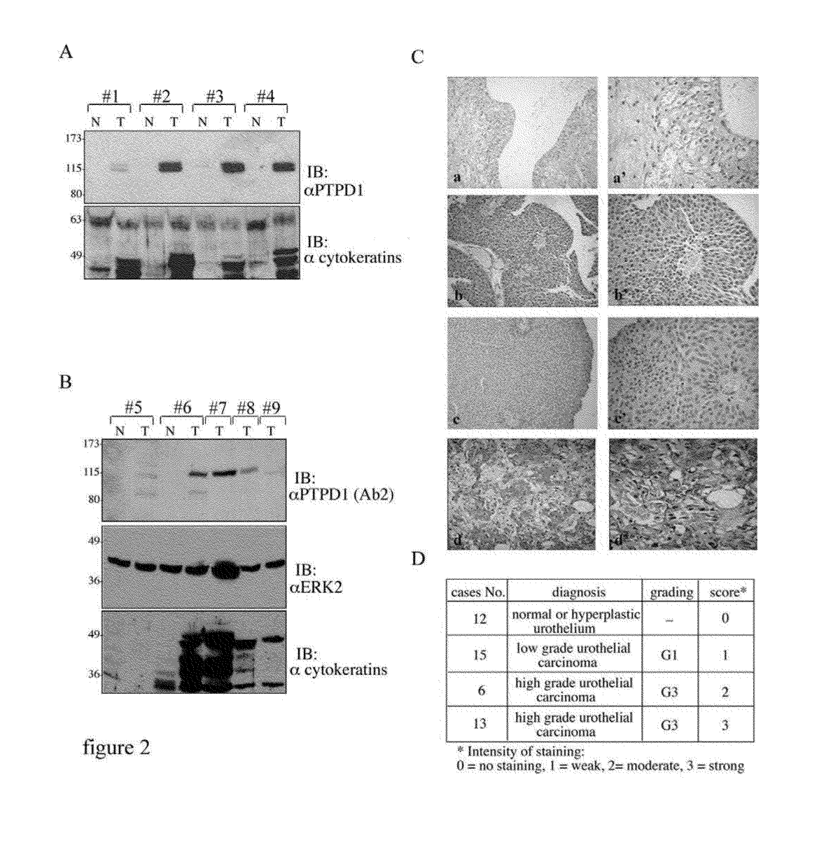

PTPD1 is over-expressed in urothelial carcinomas. Authors investigated the expression profile of PTPD1 in human bladder urothelial cancers, comparing tumors with different recurrence rates and metastatic potential. The risk grade of cancer lesions was based upon the categorization provided by the WHO Bladder grading. Tissues samples were derived from first biopsies of patients with urothelial hyperplasia, urothelial papilloma and low- or high-grade urothelial carcinoma. Tissue samples were homogenized, and protein lysates were immunoblotted with anti-PTPD1 antibody. FIG. 2A shows that PTPD1 was nearly undetectable in normal bladder tissue, hyperplastic urothelium and urothelial papilloma, whereas low levels were visible in low-grade urothelial carcinoma. However, elevated PTPD1 concentrations were seen in samples derived from high-grade urothelial carcinomas. These tumors express high levels of cytokeratins, which are typical molecular markers of epithelial bladder cancer (Sanchez-Carbayo et al., 2006a; Sanchez-Carbayo et al., 2006b) (FIG. 2B). Similar findings were obtained using an anti-PTPD1 antibody raised against the aa 618-631 aa epitope peptide of SEQ ID No. 1, as previously described (Moller et al., 1994).

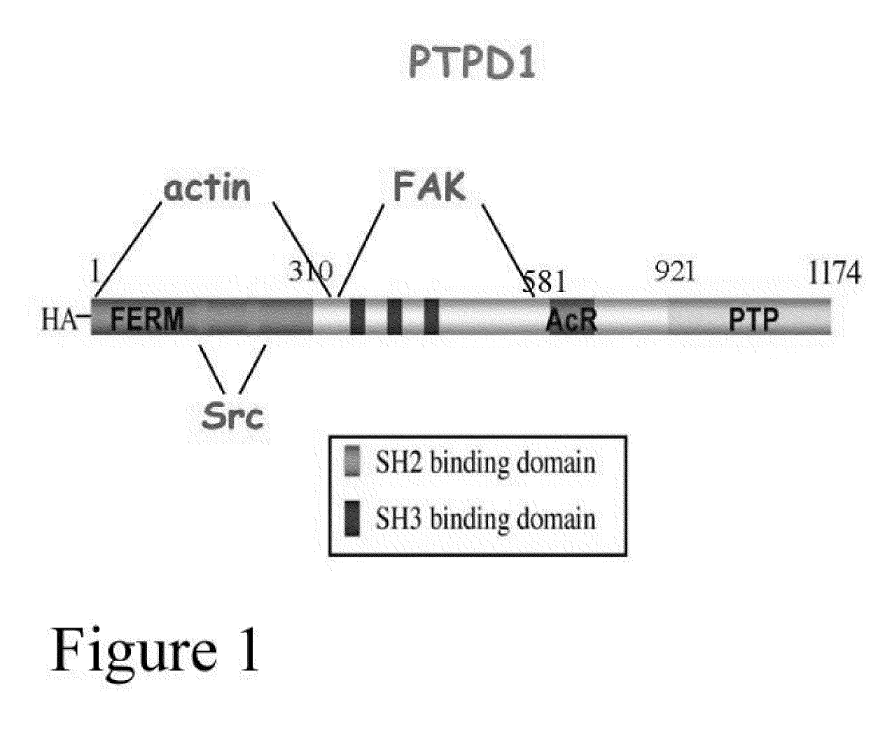

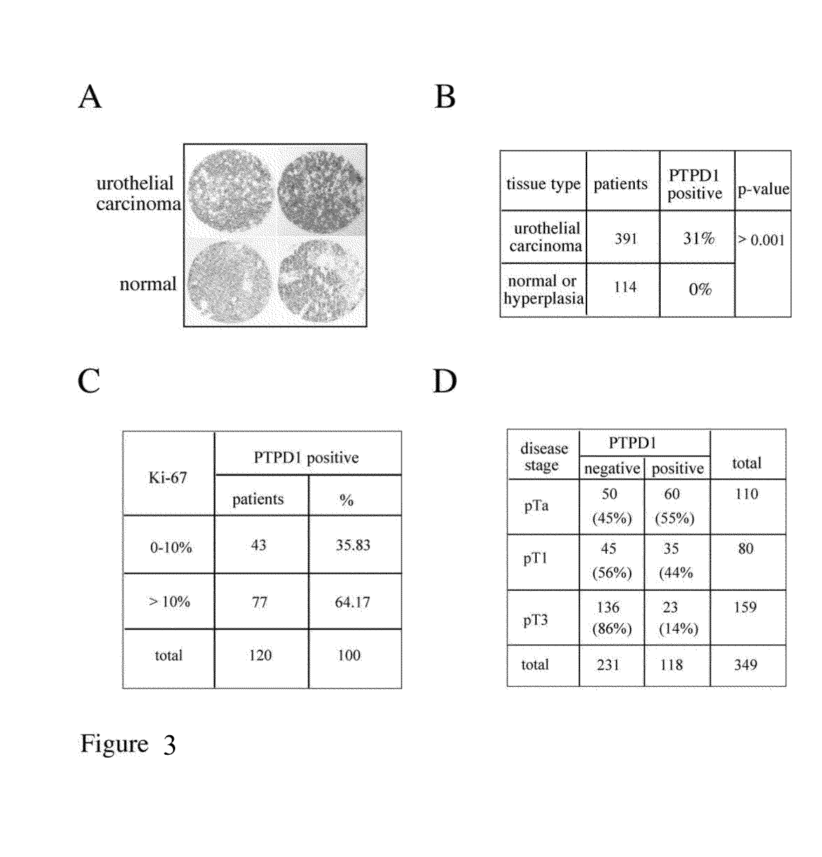

To address further this issue, authors performed immunohistochemistry on bladder sections derived from the same patients described in FIG. 1. The results shown in FIG. 2C are consistent with the immunoblotting data. PTPD1 accumulated in high-grade urothelial carcinoma, whereas PTPD1 levels were low- to nearly absent- in other tissue samples (normal urothelium, urothelial hyperplasia and urothelial papilloma). The results of this analysis, which was performed on a total of 46 patients, are summarized in FIG. 2D. Authors also evaluated PTPD1 expression in a large number of human bladder cancers by tissue microarray analysis (TMA). The array contained about 500 bladder samples ranging from normal tissue, benign lesions and urothelial carcinomas. Tissue sections were subjected to immunohistochemistry using two different anti-PTPD1 antibodies that gave similar results. FIG. 3A shows over-expression of PTPD1 in two representative urothelial carcinomas, compared to normal bladder tissues. Quantitative analysis (FIG. 3B) shows that PTPD1 was over-expressed in about 31% of bladder carcinomas, whereas a low or undetectable immunoreactive signal was obtained in other samples, including normal or hyperplastic urothelium. Ki-67 is a proliferative marker and its cutoff value of 10% is commonly used as predictive parameter of bladder cancer recurrence and progression (Blanchet et al., 2001; Liedberg et al., 2008). As shown in FIG. 3C, a greater number of PTPD1-positive sections were evident in malignant lesions with a Ki-67 cutoff value >10%, compared to those lesions with a Ki-67 value <10 (64% versus 36%, respectively). Next, authors assessed whether PTPD1 immunoreactivity correlates with the staging of bladder disease. As shown in FIG. 3D, urothelial bladder cancers in an early developmental stage (pTa) include more PTPD1-positive cells (60%) compared to cancers in intermediate (pT1) (35%) or advanced (pT3) (23%) disease stages. The inverse correlation between PTPD1 expression and disease progression might reflect a requirement of PTPD1 in an early step of tumor progression when cells first acquire a high proliferative rate and a more invasive behavior.

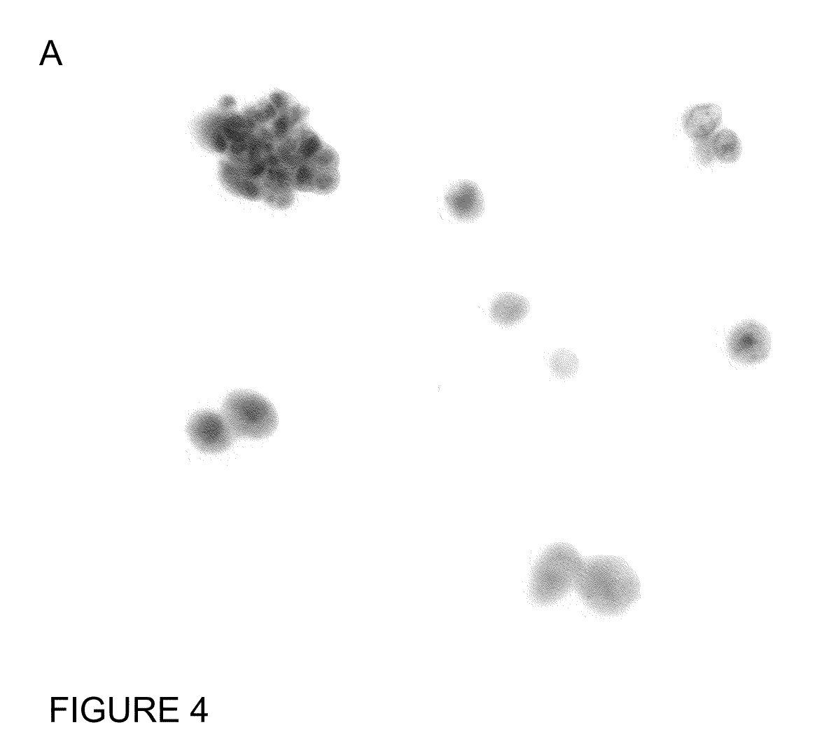

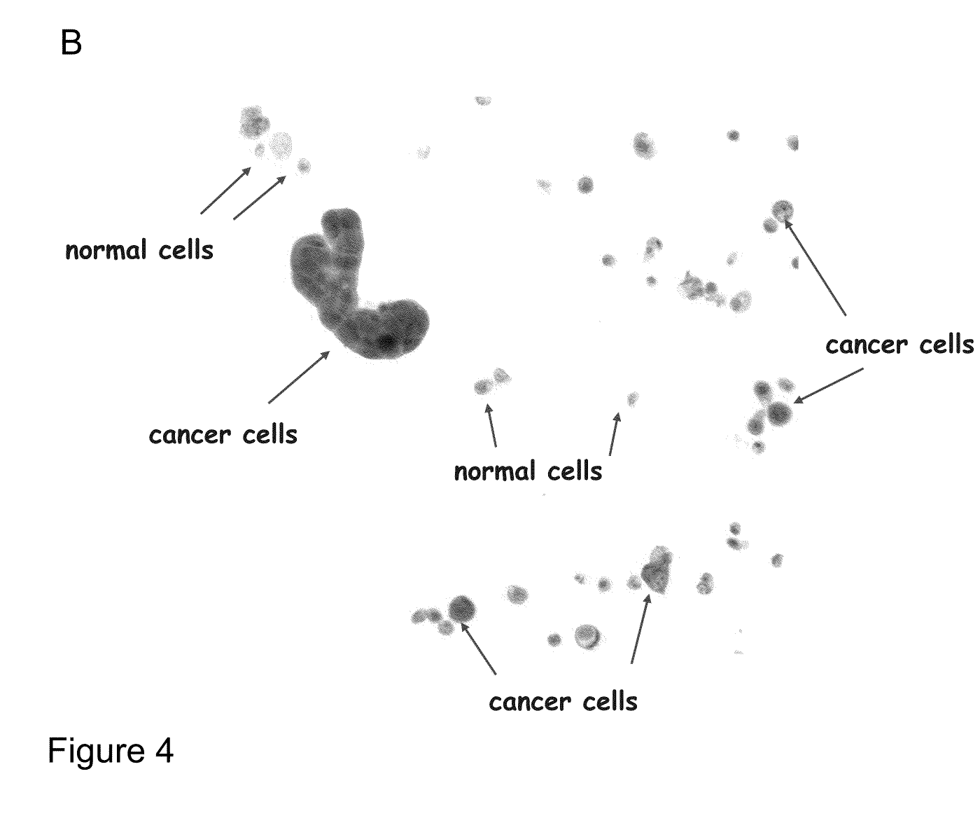

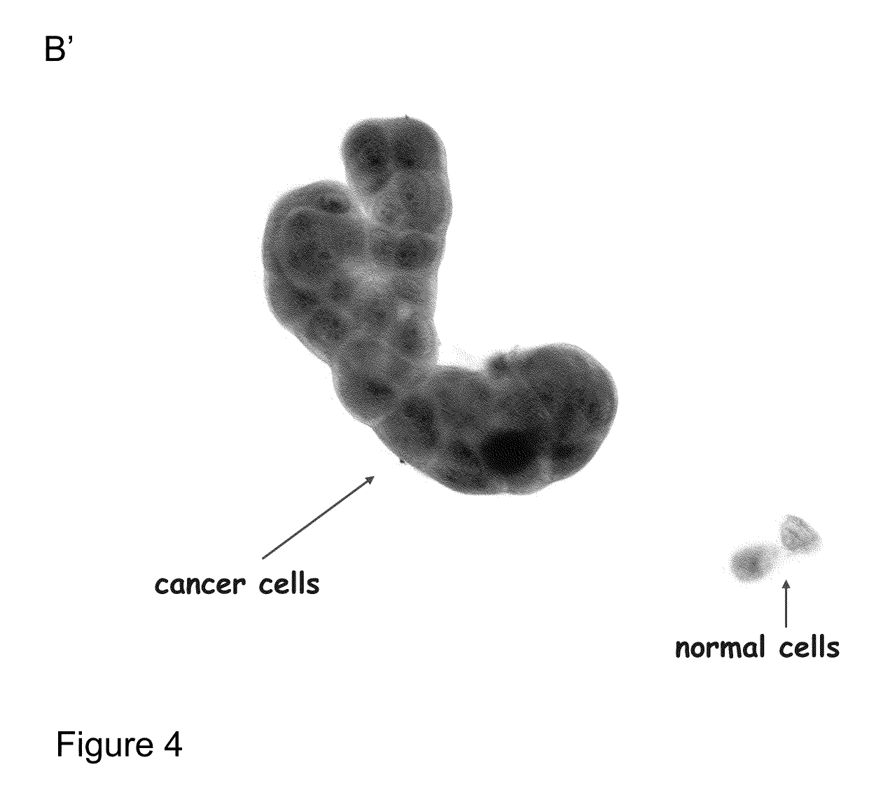

Next, authors sought to analyze PTPD1 levels in exfoliated bladder cells. Cells from urine specimens of control and bladder cancer patients were subjected to immunocytochemistry using anti-PTPD1 antibody. A representative experiment of PTPD1 detection in urine samples from bladder cancer patients is shown in FIG. 4. PTPD1 was nearly undetectable in exfoliated, normal bladder cells from healthy volunteers (FIG. 4A). In contrast, urine cancer cells from bladder patients show a strong PTPD1 immunoreactive signal, compared to normal cells present in the same urine specimen where PTPD1 staining was undetectable (FIG. 4B). Thus, PTPD1 detection in urine samples represents a novel and reliable marker for non-invasive diagnosis of aggressive bladder cancer.

### REFERENCES

Cardone, L., Carlucci, A., Affaitati, A., Livigni, A., DeCristofaro, T., Garbi, C., Varrone, S., Ullrich, A., Gottesman, M. E., Avvedimento, E. V., and Feliciello, A. (2004). Mitochondrial AKAP121 binds and targets protein tyrosine phosphatase D1, a novel positive regulator of src signaling. Mol Cell Biol 24, 4613-4626.
Carlucci, A., Gedressi, C., Lignitto, L., Nezi, L., Villa-Moruzzi, E., Avvedimento, E. V., Gottesman, M., Garbi, C., and Feliciello, A. (2008). Protein-tyrosine phosphatase PTPD1 regulates focal adhesion kinase autophosphorylation and cell migration. J Biol Chem 283, 10919-10929.
Dorner, C., Ciossek, T., Muller, S., Moller, P. H., Ullrich, A., and Lammers, R. (1998). Characterization of KIF1C, a new kinesin-like protein involved in vesicle transport from the Golgi apparatus to the endoplasmic reticulum. J Biol Chem 273, 20267-20275.
Moller, N. P., Moller, K. B., Lammers, R., Kharitonenkov, A., Sures, I., and Ullrich, A. (1994). Src kinase associates with a member of a distinct subfamily of protein-tyrosine phosphatases containing an ezrin-like domain. Proc Natl Acad Sci U S A 91, 7477-7481.

Sanchez-Carbayo, M., Socci, N. D., Lozano, J., Saint, F., and Cordon-Cardo, C. (2006a). Defining molecular profiles of poor outcome in patients with invasive bladder cancer using oligonucleotide microarrays. J Clin Oncol 24, 778-789.

Sanchez-Carbayo, M., Socci, N. D., Lozano, J. J., Haab, B. B., and Cordon-Cardo, C. (2006b). Profiling bladder cancer using targeted antibody arrays. Am J Pathol 168, 93-103.

1-16. (canceled)

17. A method for diagnosing, or the monitoring control for therapy of, bladder cancer, said method comprising detecting (i) PTPD1 protein or an immunological fragment thereof, (ii) PTPD1 enzymatic activity, or (iii) PTPD1 mRNA in a body sample.

18. The method of claim 17, wherein the detecting of the PTPD1 protein or of an immunological fragment thereof comprises allowing the body sample to react with a PTPD1 protein specific ligand to form a complex; and detecting the complex.

19. The method of claim 18, wherein the PTPD1 protein specific ligand comprises an anti-PTPD1 antibody or a derivative thereof.

20. The method of claim 19, wherein the anti-PTPD1 antibody or a derivative thereof is obtained by using as an immunogen the whole PTPD1 protein of SEQ ID No. 1 or an immunogenic fragment thereof.

21. The method of claim of 20 wherein the immunogenic fragment of the PTPD1 protein of SEQ ID No. 1 comprises between aa 751 and aa 910 of SEQ ID No. 1.

22. The method of claim 21, wherein the immunogenic fragment of the PTPD1 protein of SEQ ID No. 1 is a sequence between aa 751 and aa 910 of SEQ ID No. 1.

23. The method of claim 18, wherein the detecting step comprises detection of a specific fluorescent signal.

24. The method of claim 17, wherein the detecting of the PTPD1 enzymatic activity comprises performance by fluorescent or radiolabeled assays.

25. The method of claim 17, wherein the detecting of the PTPD1 mRNA comprises performance by Northern blot analysis, polymerase chain reaction (PCR) or PCR-derived methods, or nucleic acid amplification based methods.

26. The method of claim 17, wherein the body sample is a bladder tissue.

27. The method of claim 17, wherein the body sample is a body fluid or a fraction thereof.

28. The method of claim 27, wherein the body fluid is urine or its sediment.

29. A bladder cancer diagnostic kit comprising:
a solid phase adhered PTPD1 protein specific ligand;
a detection ligand able to bind to PTPD1 protein specific ligand associated with PTPD1 protein.

30. The bladder cancer diagnostic kit of claim 29, wherein the PTPD1 protein specific ligand is an anti-PTPD1 antibody or a derivative thereof.

31. The bladder cancer diagnostic kit of claim 30, wherein the anti-PTPD1 antibody or an active fragment thereof is obtained by using a fragment of the PTPD1 protein as antigen.

32. The bladder cancer diagnostic kit of claim 31, wherein the antigen fragment of the PTPD1 protein is comprised in the region between aa. 618 and aa. 910 of the PTPD1 protein.


SEQUENCE LISTING (1 sequence)

SEQ ID NO: 1 - protein, 1174 aa, Homo sapiens [sequence omitted]